(19) United States
(12) Patent Application Publication
(10) Pub. No.: US 2013/0289434 A1
(43) Pub. Date: Oct. 31, 2013

(54) Device for Measuring and Analyzing Electromyography signals

(71) Applicants: Willy CHOU, Tainan City (TW); Bor-Shyh Lin, Tainan City (TW); Chien-Zhi Ou, Tainan City (TW)

(72) Inventors: Willy CHOU, Tainan City (TW); Bor-Shyh Lin, Tainan City (TW); Chien-Zhi Ou, Tainan City (TW)

(73) Assignee: NATIONAL CHIAO TUNG UNIVERSITY, Hsinchu City (TW)

(21) Appl. No.: 13/757,922

(22) Filed: Feb. 4, 2013

(30) Foreign Application Priority Data
Apr. 30, 2012 (TW) 101115347

(51) Int. Cl. A61B 5/0488; A61B 5/00; A61B 5/04

(52) U.S. Cl. CPC A61B 5/04884; A61B 5/04012; A61B 5/0004; A61B 5/4205
USPC 600/546

(57) ABSTRACT

A device is for measuring and analyzing electromyography signals obtained from a target body part that is divided into an unhealthy-side muscle group and a healthy-side muscle group, and includes an unhealthy-side measuring electrode unit to be disposed on the unhealthy-side muscle group for measuring an electromyography signal therefrom so as to generate unhealthy-side measured data, a healthy-side measuring electrode unit to be disposed on the healthy-side muscle group for measuring an electromyography signal therefrom so as to generate healthy-side measured data, and a control unit determining a correlation between the unhealthy-side measured data and the healthy-side measured data.

### CROSS-REFERENCE TO RELATED APPLICATION

This application claims priority of Taiwanese Patent Application No. 101115347, filed on Apr. 30, 2012.

### BACKGROUND OF THE INVENTION

1. Field of the Invention

The present invention relates to a device for measuring and analyzing electromyography signals, more particularly to a device for measuring electromyography signals from muscle groups and analyzing a correlation between the electromyography signals.

2. Description of the Related Art

Videofluoroscopic swallow study (VFSS) is usually used for diagnosis and evaluation of a syndrome of swallowing disorder (dysphagia). However, since a patient who undergoes VFSS must be exposed to radiation, it is not suitable for the patent to have another VFSS within a short amount of time. Moreover, VFSS must be performed by a specialist, and is thus unsuitable for the patient to perform VFSS at home.

Fiberoptic endoscopic examination of swallowing is another method commonly used for assessment of swallowing disorder. During the assessment, a fiberoptic endoscope is inserted into a patient's nostril and placed on the hypopharynx for observing movements of the patient's hypopharynx, throat, and upper part of trachea. However, since this method is an invasive assessment method and requires a bulky equipment, this method is also not suitable to regularly evaluate an effect of rehabilitation practice associated with swallowing disorder for a patient.

Therefore, how to develop a non-invasive measuring and analyzing equipment which facilitates regular examination and evaluation of a patient with swallowing disorder is a subject of endeavor in the present invention.

### SUMMARY OF THE INVENTION

Therefore, an object of the present invention is to provide a device for measuring and analyzing electromyography signals which makes it easy for a user to examine and evaluate muscle dysfunction.

Accordingly, the device, according to the present invention, is adapted for measuring and analyzing electromyography signals obtained from a target body part that is divided into an unhealthy-side muscle group and a healthy-side muscle group. The device comprises an unhealthy-side measuring electrode unit, a healthy-side measuring electrode unit and a control unit. The unhealthy-side measuring electrode unit is adapted to be disposed on the unhealthy-side muscle group and is adapted for measuring an electromyography signal from the unhealthy-side muscle group so as to generate unhealthy-side measured data. The healthy-side measuring electrode unit is adapted to be disposed on the healthy-side muscle group and is adapted for measuring an electromyography signal from the healthy-side muscle group so as to generate healthy-side measured data. The control unit is connected electrically to the unhealthy-side measuring electrode unit and the healthy-side measuring electrode, and determines a correlation between the unhealthy-side measured data and the healthy-side measured data.

Effects of the present invention reside in that, by virtue of the non-invasive unhealthy-side and healthy-side measuring electrode units adapted for measuring the electromyography signals from the target body part, regular examination for a patient with swallowing disorder may be facilitated. Besides, by virtue of the control unit which determines a correlation between the unhealthy-side measured data and the healthy-side measured data, a user is able to evaluate severity of swallowing disorder of the patient based on a degree of the correlation.

### BRIEF DESCRIPTION OF THE DRAWINGS

Other features and advantages of the present invention will become apparent in the following detailed description of three preferred embodiments with reference to the accompanying drawings, of which:

FIG. 1 is a block diagram of a first preferred embodiment of a device, according to the present invention, for measuring and analyzing electromyography signals;

FIG. 2 is a schematic diagram of the first preferred embodiment used to measure a subject;

FIGS. 3 to 6 are electromyograms of right-side orbicularis oris (OO) muscles, right-side masseter (MS) muscles, right-side submental (SUB) muscles and right-side laryngeal strap (LS) muscles, respectively of a subject with normal swallowing;

FIGS. 7 to 10 are electromyograms of left-side OO muscles, left-side MS muscles, left-side SUB muscles and left-side LS muscles, respectively of the subject with normal swallowing;

FIGS. 11 to 14 are electromyograms of right-side OO muscles, right-side MS muscles, right-side SUB muscles and right-side LS muscles, respectively of a subject with swallowing disorder;

FIGS. 15 to 18 are electromyograms of left-side OO muscles, left-side MS muscles, left-side SUB muscles and left-side LS muscles, respectively of the subject with swallowing disorder; and

FIG. 19 is a perspective view of a third preferred embodiment of the device, according to the present invention, for measuring and analyzing electromyography signals.

### DETAILED DESCRIPTION OF THE PREFERRED EMBODIMENTS

Before the present invention is described in greater detail with reference to the preferred embodiments, it should be noted that the same reference numerals are used to denote the same elements throughout the following description.

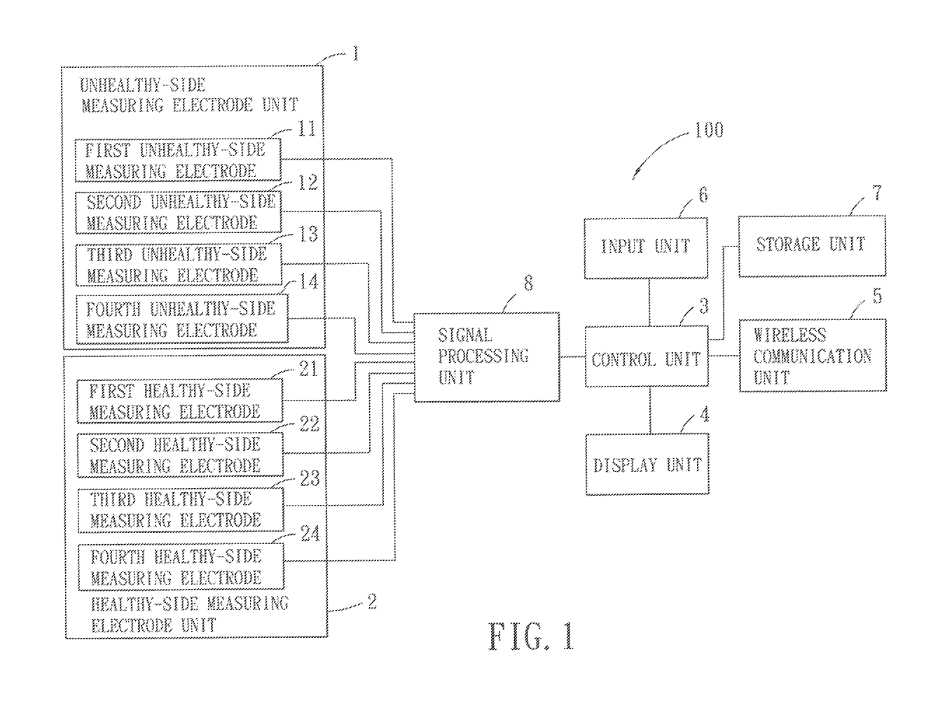

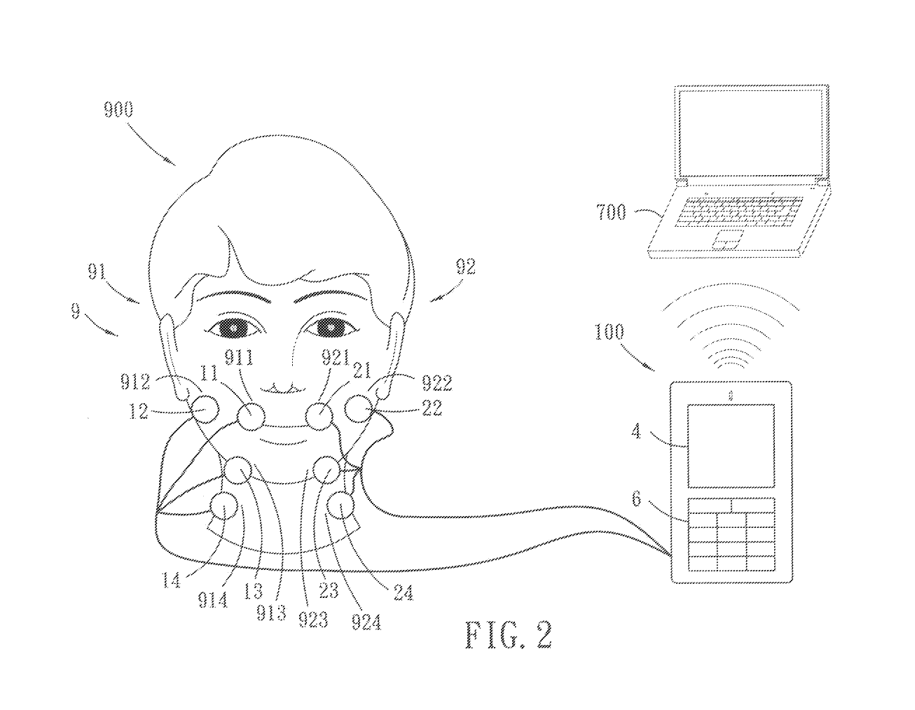

Referring to FIGS. 1 and 2, the first preferred embodiment of a device, according to the present invention, for measuring and analyzing electromyography signals is illustrated. The device 100 for measuring and analyzing electromyography signals is utilized to measure electromyography signals obtained from a target body part 9 of a subject 900. In this embodiment, the target body part 9 is a muscle group associated with swallowing behavior, and the device 100 is used to measure the electromyography signals of the target body part 9 resulting from the swallowing behavior. The target body part 9 is divided into an unhealthy-side muscle group 91 and a healthy-side muscle group 92. In other words, one side of the target body part 9 of the subject 900 is healthy and the other side thereof has a syndrome of swallowing disorder. The unhealthy-side muscle group 91 includes unhealthy-side orbicularis oris (OO) muscles 911, unhealthy-side masseter (MS) muscles 912, unhealthy-side submental (SUB) muscles 913 and unhealthy-side laryngeal strap (LS) muscles 914. The healthy-side muscle group 92 includes healthy-side orbicularis oris (OO) muscles 921, healthy-side masseter (MS) muscles 922, healthy-side submental (SUB) muscles 923 and healthy-side laryngeal strap (LS) muscles 924.

The device 100 for measuring and analyzing electromyography signals comprises a control unit 3, and a signal processing unit 8, a display unit 4, a wireless communication unit 5, an input unit 6 and a storage unit 7, each of which is coupled electrically to the control unit 3. The device 100 further comprises an unhealthy-side measuring electrode unit 1 and a healthy-side measuring electrode unit 2, each of which is coupled electrically to the signal processing unit 8.

The unhealthy-side measuring electrode unit 1 is adapted to be disposed on the unhealthy-side muscle group 91 and is adapted for measuring an electromyography signal from the unhealthy-side muscle group 91 so as to generate unhealthy-side measured data. The unhealthy-side measuring electrode unit 1 includes a first unhealthy-side measuring electrode 11, a second unhealthy-side measuring electrode 12, a third, unhealthy-side measuring electrode 13 and a fourth unhealthy-side measuring electrode 14, each of which is to be disposed on a respective one of the unhealthy-side OO muscles 911, the unhealthy-side MS muscles 912, the unhealthy-side SUB muscles 913 and the unhealthy-side LS muscles 914, and each of which is for measuring an electromyography signal of a respective one of the unhealthy-side OO muscles 911, the unhealthy-side MS muscles 912, the unhealthy-side SUB muscles 913 and the unhealthy-side LS muscles 914, so as to generate a respective one of first unhealthy-side measured sub-data, second unhealthy-side measured sub-data, third unhealthy-side measured sub-data and fourth unhealthy-side measured sub-data of the unhealthy-side measured data. It is noted that, each of the first to fourth unhealthy-side measuring electrodes 11-14 measures the electromyography signal at a preset frequency, and each of the first to fourth unhealthy-side measured sub-data thus generated includes a plurality of electromyography signals which are sorted in an order the electromyography signals are measured. The first unhealthy-side measured sub-data, the second unhealthy-side measured sub-data, the third unhealthy-side measured sub-data and the fourth unhealthy-side measured sub-data are respectively represented as X₁, X₂, X₃ and X₄, and X₁={X₁₁, X₁₂, . . . , X₁ₐ}, X₂={X₂₁, X₂₂, . . . , X_{2b}}, X₃={X₃₁, X₃₂, . . . , X_{3c}} and X₄={X₄₁, X₄₂, . . . , X_{4d}}.

The healthy-side measuring electrode unit 2 is adapted to be disposed on the healthy-side muscle group 92 and is adapted for measuring an electromyography signal from the healthy-side muscle group 92 so as to generate healthy-side measured data. The healthy-side measuring electrode unit 2 includes a first healthy-side Measuring electrode 21, a second healthy-side measuring electrode 22, a third healthy-side measuring electrode 23 and a fourth healthy-side measuring electrode 24, each of which is to be disposed on a respective one of the healthy-side OO muscles 921, the healthy-side MS muscles 922, the healthy-side SUB muscles 923 and the healthy-side LS muscles 924, and each of which is for measuring an electromyography signal of a respective one of the healthy-side OO muscles 921, the healthy-side MS muscles 922, the healthy-side SUB muscles 923 and the healthy-side LS muscles 924, so as to generate a respective one of first healthy-side measured sub-data, second healthy-side measured sub-data, third healthy-side measured sub-data and fourth healthy-side measured sub-data of the healthy-side measured data. It is noted that, each of the first to fourth healthy-side measuring electrodes 21-24 measures the electromyography signal at a preset frequency, and each of the first to fourth healthy-side measured sub-data thus generated includes a plurality of electromyography signals which are sorted in an order the electromyography signals are measured. The first healthy-side measured sub-data, the second healthy-side measured, sub-data, the third healthy-side measured sub-data and the fourth healthy-side measured sub-data are respectively represented as Y₁, Y₂, Y₃, and Y₄, and Y₁={Y₁₁, Y₁₂, . . . , Y₁ₐ}, Y₂={Y₂₁, Y₂₂, . . . , Y_{2b}}, Y₃={Y₃₁, Y₃₂, . . . , Y_{3c}} and Y₄={Y₄₁, Y₄₂, . . . , Y_{4d}}.

The signal processing unit 8 receives the electromyography signals measured by the unhealthy-side measuring electrode unit 1 and the healthy-side measuring electrode unit 2, filters and amplifies the same, and transmits the electromyography signals thus filtered and amplified to the control unit 3. The control unit 3 determines a correlation between the unhealthy-side measured data and the healthy-side measured data such that severity of swallowing disorder of the subject 900 may be evaluated based on the correlation. When the correlation between the unhealthy-side measured data and the healthy-side measured data is lower, it means that consistency of the swallowing behavior between the unhealthy-side muscle group 91 and the healthy-side muscle group 92 is lower, and the syndrome of swallowing disorder of the subject 900 is more severe. On the other hand, when the correlation between the unhealthy-side measured data and the healthy-side measured data is higher, it means that consistency of the swallowing behavior between the unhealthy-side muscle group 91 and the healthy-side muscle group 92 is higher, and the syndrome of swallowing disorder of the subject 900 is milder.

In this embodiment, the Pearson's correlation coefficient is adopted to represent the correlation. The Pearson's correlation coefficient is given a value between +1 and −1. When the value is closer to +1, it means that a positive linear correlation is higher; when the value is closer to −1, it means that a negative linear correlation is higher; and when the value is closer to 0, it means that the correlation is lower. Calculations associated with the Pearson's correlation coefficient in this embodiment are illustrated hereinafter.

The control unit 3 is configured to perform integration operations on the first unhealthy-side measured sub-data X₁ and the first healthy-side measured sub-data Y₁ so as to obtain respectively first unhealthy-side integrated data Xₓ₁ and first healthy-side integrated data Yₓ₁, wherein

$${X}_{I 1} = \{ {X}_{11} , {X}_{11} + {X}_{12} , … , {\sum }_{i = 1}^{a} {X}_{1 i} \} and$$

$${Y}_{I 1} = \{ {Y}_{11 ,} {Y}_{11} + {Y}_{12} , … , {\sum }_{i = 1}^{a} {Y}_{1 i} \} .$$

Subsequently, the control unit 3 is further configured to determine a correlation between the first unhealthy-side integrated data Xₓ₁ and the first healthy-side integrated data Yₓ₁. The Pearson's correlation coefficient therebetween is obtained by the following equation:

$${r}_{{X}_{i 1} {y}_{i 1}} = \frac{{\sum }_{i = 1}^{a} ( {X}_{1 i} - \overline{{X}_{I 1}} ) ( {Y}_{1 i} - \overline{{Y}_{I 1}} )}{\sqrt{{\sum }_{i = 1}^{a} {( {X}_{1 i} - \overline{{X}_{I 1}} )}^{2}} \sqrt{{\sum }_{i = 1}^{a} {( {Y}_{1 i} - \overline{{Y}_{I 1}} )}^{2}}} .$$

By virtue of the Pearson's correlation coefficient between the first unhealthy-side integrated data Xₓ₁ and the first healthy-side integrated data Yₓ₁, severity of swallowing disorder associated with the unhealthy-side OO muscles 911 may be represented.

Similarly, the control unit 3 is configured to perform integration operations on the second unhealthy-side measured sub-data X₂ and the second healthy-side measured sub-data Y₂ so as to obtain respectively second unhealthy-side integrated data Xₓ₂ and second healthy-side integrated data Yₓ₂, wherein

$${X}_{I 2} = \{ {X}_{21} , {X}_{21} + {X}_{22} , … , {\sum }_{i = 1}^{b} {X}_{2 i} \} and$$

$${Y}_{I 2} = \{ {Y}_{21 ,} {Y}_{21} + {Y}_{22} , … , {\sum }_{i = 1}^{b} {Y}_{2 i} \} .$$

Subsequently, the control unit 3 is further configured to determine a correlation between the second unhealthy-side integrated data Xₓ₂ and the second healthy-side integrated data Yₓ₂. The Pearson's correlation coefficient therebetween is obtained by the following equation;

$${r}_{{X}_{i 2} {y}_{i 2}} = \frac{{\sum }_{i = 1}^{b} ( {X}_{2 i} - \overline{{X}_{I 2}} ) ( {Y}_{2 i} - \overline{{Y}_{I 2}} )}{\sqrt{{\sum }_{i = 1}^{b} {( {X}_{2 i} - \overline{{X}_{I 2}} )}^{2}} \sqrt{{\sum }_{i = 1}^{b} {( {Y}_{2 i} - \overline{{Y}_{I 2}} )}^{2}}} .$$

By virtue of the Pearson's correlation coefficient between the second unhealthy-side integrated data Xₓ₂ and the second healthy-side integrated data Yₓ₂, severity of swallowing disorder associated with the unhealthy-side MS muscles 912 may be represented.

Likewise, the control unit 3 is configured to perform integration operations on the third unhealthy-side measured sub-data X₃ and the third healthy-side measured sub-data Y₃ so as to obtain respectively third unhealthy-side integrated data Xₓ₃ and third healthy-side integrated data Yₓ₃, wherein

$${X}_{I 3} = \{ {X}_{31} , {X}_{31} + {X}_{32} , … , {\sum }_{i = 1}^{c} {X}_{3 i} \} and$$

$${Y}_{I 3} = \{ {Y}_{31 ,} {Y}_{31} + {Y}_{32} , … , {\sum }_{i = 1}^{c} {Y}_{3 i} \} .$$

Subsequently, the control unit 3 is further configured to determine a correlation between the third unhealthy-side integrated data Xₓ₃ and the third healthy-side integrated data Yₓ₃. The Pearson's correlation coefficient therebetween is obtained by the following equation:

$${r}_{{X}_{i 3} {y}_{i 3}} = \frac{{\sum }_{i = 1}^{c} ( {X}_{3 i} - \overline{{X}_{I 3}} ) ( {Y}_{3 i} - \overline{{Y}_{I 3}} )}{\sqrt{{\sum }_{i = 1}^{c} {( {X}_{3 i} - \overline{{X}_{I 3}} )}^{2}} \sqrt{{\sum }_{i = 1}^{c} {( {Y}_{3 i} - \overline{{Y}_{I 3}} )}^{2}}} .$$

By virtue of the Pearson's correlation coefficient between the third unhealthy-side integrated data Xₓ₃ and the third healthy-side integrated data Yₓ₃, severity of swallowing disorder associated with the unhealthy-side SUB muscles 913 may be represented.

Furthermore, the control unit 3 is configured to perform integration operations on the fourth unhealthy-side measured sub-data X₄ and the fourth healthy-side measured sub-data Y₄ so as to obtain respectively fourth unhealthy-side integrated data Xₓ₄ and fourth healthy-side integrated data Yₓ₄, wherein

$${X}_{I 4} = \{ {X}_{41} , {X}_{41} + {X}_{42} , … , {\sum }_{i = 1}^{d} {X}_{4 i} \} and$$

$${Y}_{I 4} = \{ {Y}_{41 ,} {Y}_{41} + {Y}_{42} , … , {\sum }_{i = 1}^{d} {Y}_{4 i} \} .$$

Subsequently, the control unit 3 is further configured to determine a correlation between the fourth unhealthy-side integrated data Xₓ₄ and the fourth healthy-side integrated data Yₓ₄. The Pearson's correlation coefficient therebetween is obtained by the following equation:

$${r}_{{X}_{i 4} {y}_{i 4}} = \frac{{\sum }_{i = 1}^{d} ( {X}_{4 i} - \overline{{X}_{I 4}} ) ( {Y}_{4 i} - \overline{{Y}_{I 4}} )}{\sqrt{{\sum }_{i = 1}^{d} {( {X}_{4 i} - \overline{{X}_{I 4}} )}^{2}} \sqrt{{\sum }_{i = 1}^{d} {( {Y}_{4 i} - \overline{{Y}_{I 4}} )}^{2}}} .$$

By virtue of the Pearson's correlation coefficient between the fourth unhealthy-side integrated data Xₓ₄ and the fourth healthy-side integrated data Yₓ₄, severity of swallowing disorder associated with the unhealthy-side LS muscles 914 may be represented.

Since durations of muscle contraction of each of the unhealthy-side muscle group 91 and the healthy-side muscle group 92 due to the swallowing behavior are different, and swallowing muscles with swallowing disorder generally have shorter duration of muscle contraction, the severity of swallowing disorder may be further evaluated by a correlation between the duration of muscle contraction. How to represent the severity of swallowing disorder by virtue of the correlation between the duration of muscle contraction in this embodiment is illustrated hereinafter.

The control unit 3 is configured to generate a first unhealthy-side swallow duration value t_{X1}, a second unhealthy-side swallow duration value t_{X2}, a third unhealthy-side swallow duration value t_{X3}, a fourth unhealthy-side swallow duration value t_{X4}, a first healthy-side swallow duration value t_{Y1}, a second healthy-side swallow duration value t_{Y2}, a third healthy-side swallow duration value t_{X3} and a fourth healthy-side swallow duration value t_{X4}, each of which is associated with duration of muscle contraction due to swallowing behavior, according to the first unhealthy-side measured sub-data X₁, the second unhealthy-side measured sub-data X₂, the third unhealthy-side measured sub-data X₃, the fourth unhealthy-side measured sub-data X₄, the first healthy-side measured sub-data Y₁, the second healthy-side measured sub-data Y₂, the third healthy-side measured sub-data Y₃ and the fourth healthy-side measured sub-data Y₄, respectively.

Subsequently, the control unit 3 is further configured to determine a correlation between a set tₓ of the first unhealthy-side swallow duration value t_{X1}, the second unhealthy-side swallow duration value t_{X2}, the third unhealthy-side swallow duration value t_{X3} and the fourth unhealthy-side swallow duration value t_{X4}, and a set t_{y} of the first healthy-side swallow duration value t_{Y1}, the second healthy-side swallow duration value t_{Y2}, the third healthy-side swallow duration value t_{Y3} and the fourth healthy-side swallow duration value t_{Y4}. The sets t_{X} and t_{Y} are defined as t_{X}={t_{X1}, t_{X3}, t_{X4}} and t_{Y}={t_{Y1}, t_{Y2}, t_{Y3}, t_{Y4}}. The Pearson's correlation coefficient (i.e., the aforesaid correlation) between the duration of muscle contraction of a respective one of the unhealthy-side muscle group 91 and the healthy-side muscle group 92 is obtained by the following equation;

$${r}_{{i}_{X} {i}_{Y}} = \frac{{\sum }_{i = 1}^{4} ( {t}_{Xi} - \overline{{t}_{X}} ) ( {t}_{Yi} - \overline{{t}_{Y}} )}{\sqrt{{\sum }_{i = 1}^{4} {( {t}_{Xi} - \overline{{t}_{X}} )}^{2}} \sqrt{{\sum }_{i = 1}^{d} {( {t}_{Yi} - \overline{{t}_{Y}} )}^{2}}} .$$

By virtue of the Pearson's correlation coefficient between the duration of muscle contraction of a respective one of the unhealthy-side muscle group 91 and the healthy-side muscle group 92, severity of swallowing disorder associated with the unhealthy-side muscle group 91 may be represented.

On the other hand, by virtue of determining a correlation between means of the electromyography signals of a respective one of the unhealthy-side muscle group 91 and the healthy-side muscle group 92, respectively, within the duration of muscle contraction, the severity of swallowing disorder may also be revealed. Calculations associated with the means and the correlation are described hereinafter.

The control unit 3 is configured to calculate a mean of the electromyography signals of each of the first unhealthy-side measured sub-data X₁, the second unhealthy-side measured sub-data X₂, the third unhealthy-side measured sub-data X₃, the fourth unhealthy-side measured sub-data X₄, the first healthy-side measured sub-data Y₁, the second healthy-side measured sub-data Y₂, the third healthy-side measured sub-data Y₃ and the fourth healthy-side measured sub-data Y₄, within the duration of muscle contraction, so as to generate a respective one of a first unhealthy-side measured mean X̅₁̅, a second unhealthy-side measured mean X̅₂̅, a third unhealthy-side measured mean X̅₃̅, a fourth unhealthy-side measured mean X̅₄̅, a first healthy-side measured mean Y̅₁̅, a second healthy-side measured mean Y̅₂̅, a third healthy-side measured mean Y̅₃̅ and a fourth healthy-side measured mean Y̅₄̅. It is noted that, the mean in this embodiment is obtained by calculating a mean of a plurality of absolute values of a respective one of the electromyography signals. The reason behind using the absolute values for calculations is that, since the control unit 3 is configured to subtract a base value that is associated with a static state of respective muscles from the electromyography signal thus measured, a negative result of the electromyography signal may be obtained. The absolute value of the electromyography signal stands for amplitude of the electromyography signal, i.e., the intensity of the electromyography signal.

Afterward, the control unit 3 is further configured to determine a correlation between a set X′ of the first unhealthy-side measured mean X̅₁̅, the second unhealthy-side measured mean X̅₂̅, the third unhealthy-side measured mean X̅₃̅ and the fourth unhealthy-side measured mean X̅₄̅, and a set Y′ of the first healthy-side measured mean Y̅₁̅, the second healthy-side measured mean Y̅₂̅, the third healthy-side measured mean Y̅₃̅ and the fourth healthy-side measured mean Y̅₄̅. The sets X′ and Y′ are defined as X′={ X̅₁̅, X̅₂̅, X̅₃̅, X̅₄̅} and Y′={ Y̅₁̅, Y̅₂̅, Y̅₃̅, Y̅₄̅}. The Pearson' correlation coefficient (i.e., the aforesaid correlation) between the means of the electromyography signals of a respective one of the unhealthy-side muscle group 91 and the healthy-side muscle group 92 within the duration of muscle contraction is obtained by the following equation:

$${r}_{{X}^{′} {Y}^{′}} = \frac{{\sum }_{i = 1}^{4} ( \overline{{X}_{i}} - \overline{{X}^{′}} ) ( \overline{{Y}_{i}} - \overline{{Y}^{′}} )}{\sqrt{{\sum }_{i = 1}^{4} {( \overline{{X}_{i}} - \overline{{X}^{′}} )}^{2}} \sqrt{{\sum }_{i = 1}^{d} {( \overline{{Y}_{i}} - \overline{{Y}^{′}} )}^{2}}}$$

When a result of each of the aforementioned Pearson's correlation coefficients is closer to +1, it means that the electromyography signals of the unhealthy-side muscle group 91 and the healthy-side muscle group 92 resulting from the swallowing behavior are similar. On the contrary, when the result of each of the aforementioned Pearson's correlation coefficients is much smaller than +1, it means that the electromyography signals of the unhealthy-side muscle group 91 and the healthy-side muscle group 92 resulting from the swallowing behavior are more divergent, that is, the syndrome of swallowing disorder is more severe.

Functionalities of the other components of the device 100 for measuring and analyzing electromyography signals is illustrated in the following. The input unit 6 is implemented with a plurality of keys, and alternatively may be an input device including a touch module that enables user operation so as to generate a control signal to the control unit 3 for performing various operations. The storage unit 7 is provided for storing measured data (i.e., the unhealthy-side and healthy-side measured data) and calculation results associated with the correlations. The display unit 4 is provided for displaying the measured data and the calculation results associated with the correlations in a manner of text or diagram (i.e., the electromyogram). The wireless communication unit 5 may apply at least one wireless communication technology, such as Bluetooth, ZigBee, 2G, 2.5G, 2.75G, 3G, WiFi, WiMax, infrared, radio, etc., such that the device 100 is capable of communicating with an external electronic device 700 (for example, a notebook computer) via the wireless communication unit 5 so as to transmit the measured data and the calculation results associated with the correlations to the external electronic device 700.

Data associated with measurement of the electromyography signals and determination of the correlations by the device 100 in this embodiment are provided hereinafter. The data are obtained from a subject with normal swallowing and a subject who suffers from swallowing disorder. The subject with normal swallowing is a 64-year-old male, who has no medical history of neuromuscular disorder, has not undergone head and neck surgery or radiation therapy, and is not taking medications (such as muscle relaxant) that might affect neuromuscular functions. The subject who suffers from swallowing disorder is a 65-year-old male, who has dysphagia following unilateral stroke, has high blood pressure (hypertension) and diabetes, has incidence of right brain hemorrhagic strobe over two months, and relies on nasogastric tube feeding. Clinical assessment of swallowing is described as follows. Movement range of left side lips and tongue is relatively small, lift force of left side soft palate is weak, delayed triggering of swallowing reflex, coughing & wet vice following swallowing, weak spontaneous coughing, reflexic coughing appear moderate strength, and the functional oral intake scale is 1. During the testing procedure, the subjects were instructed to drink 5 cc of water.

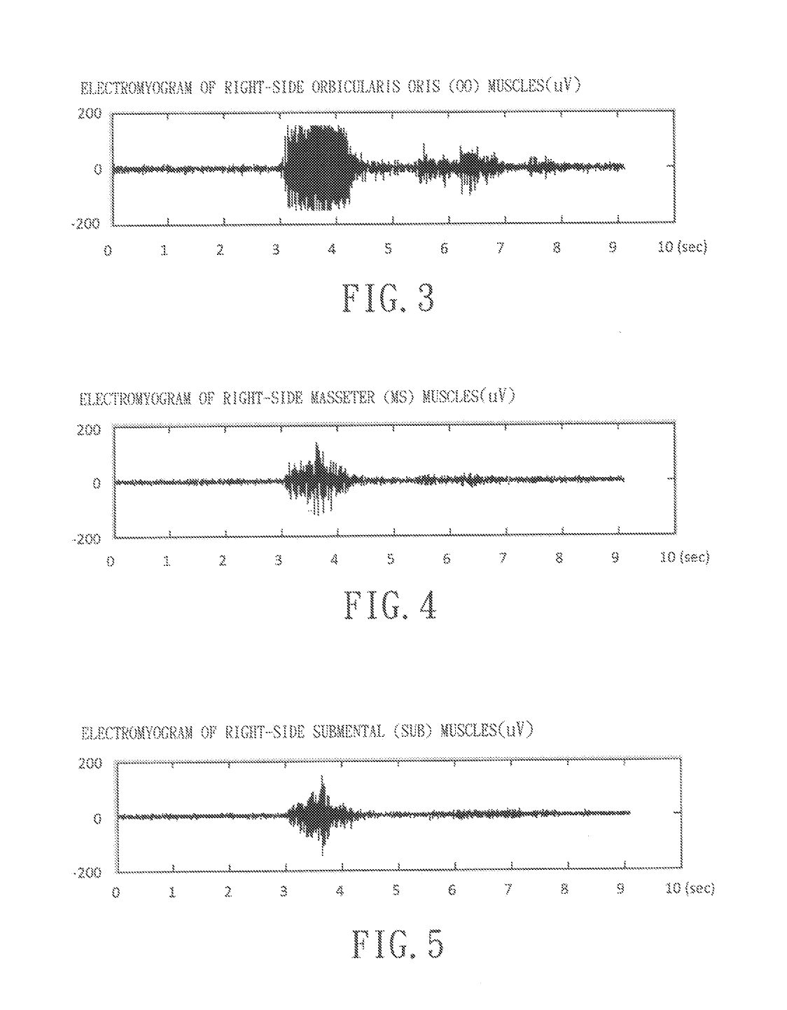

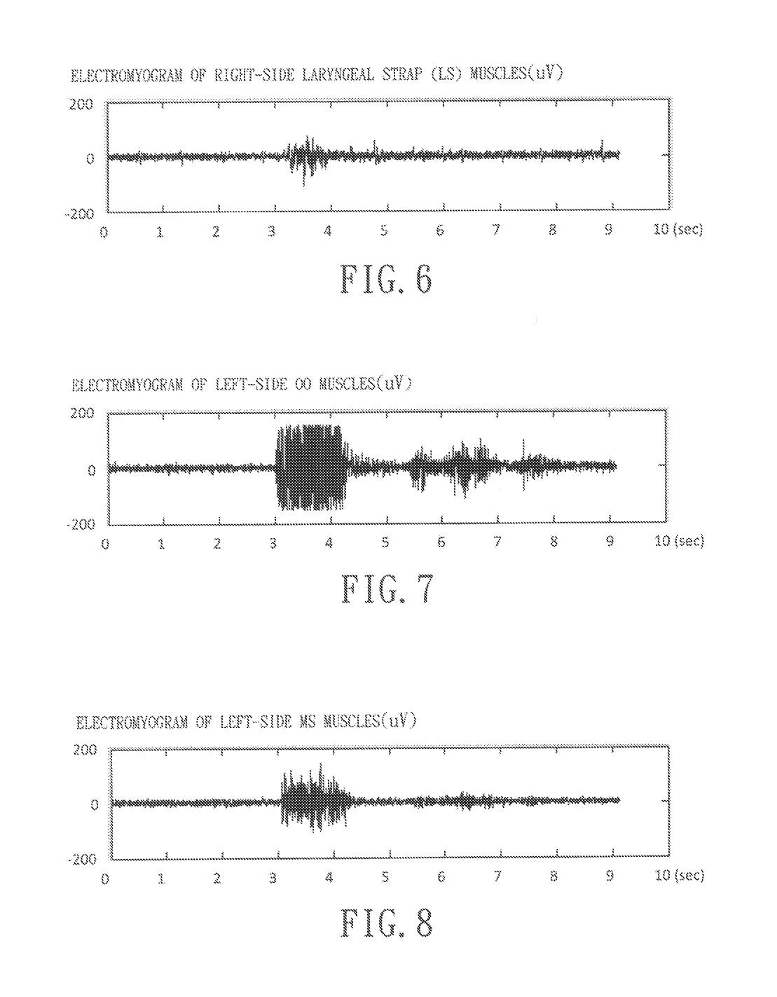

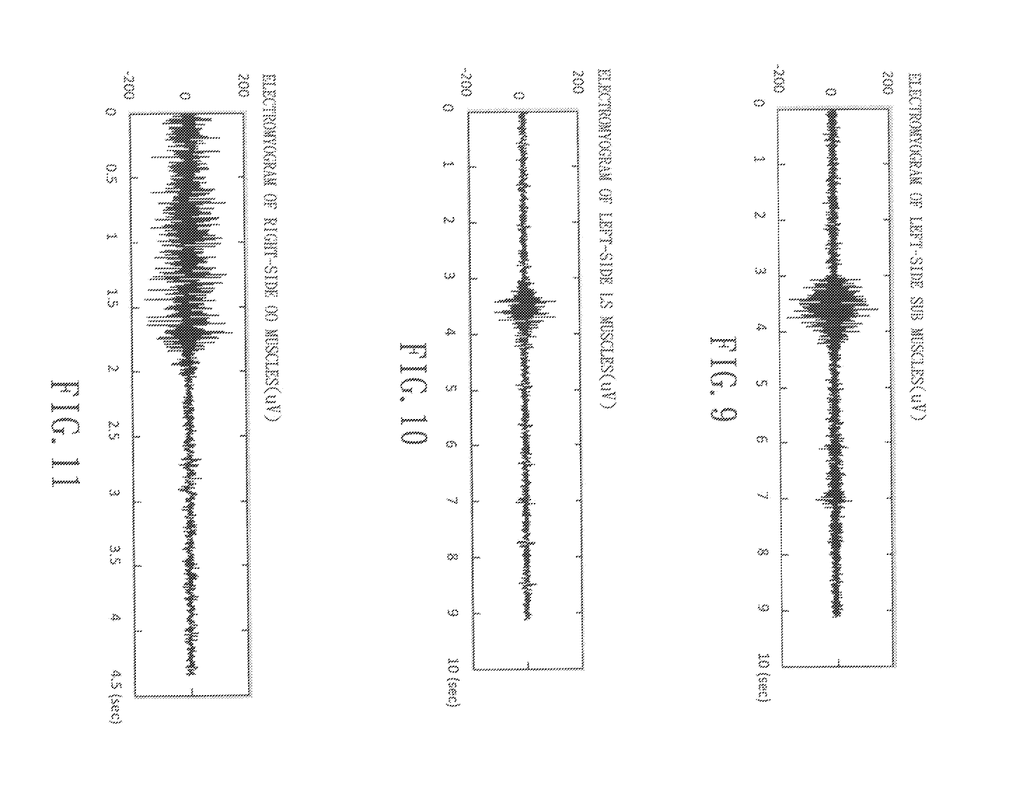

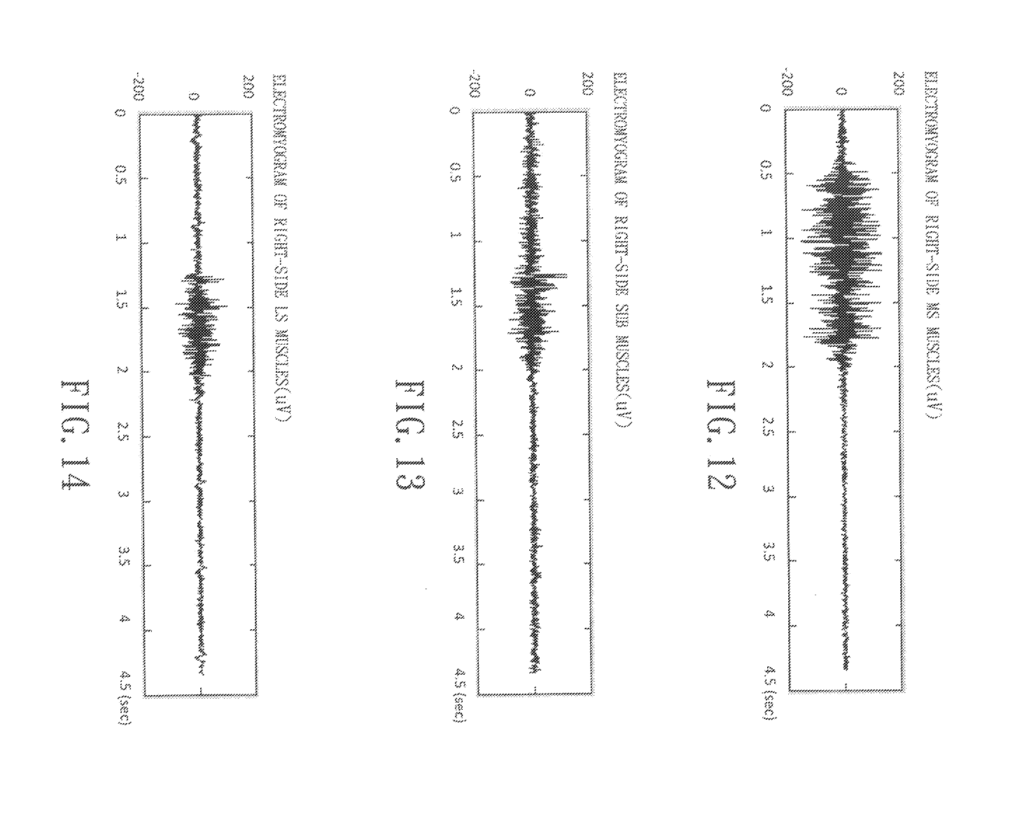

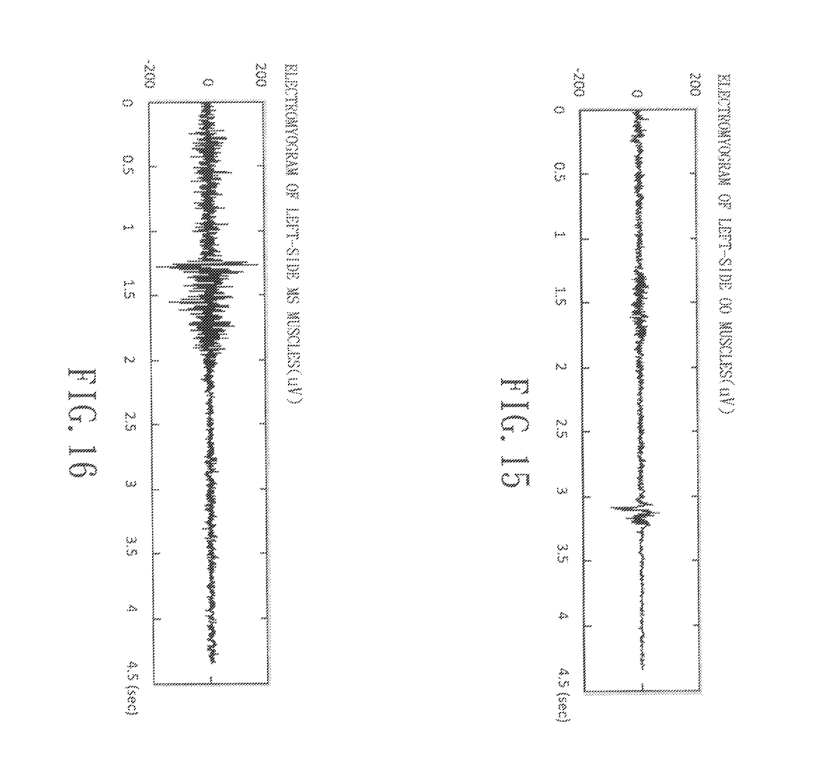

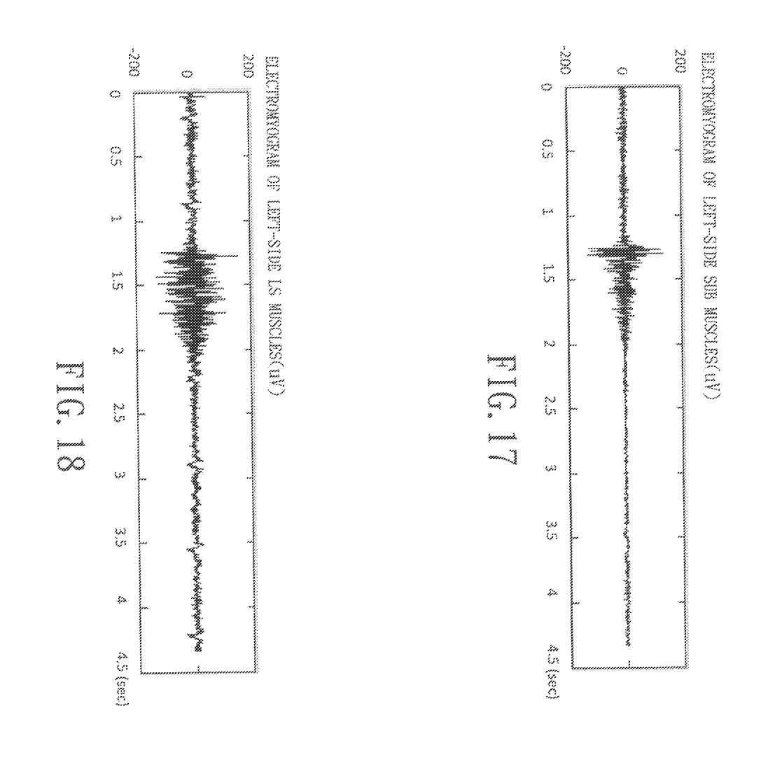

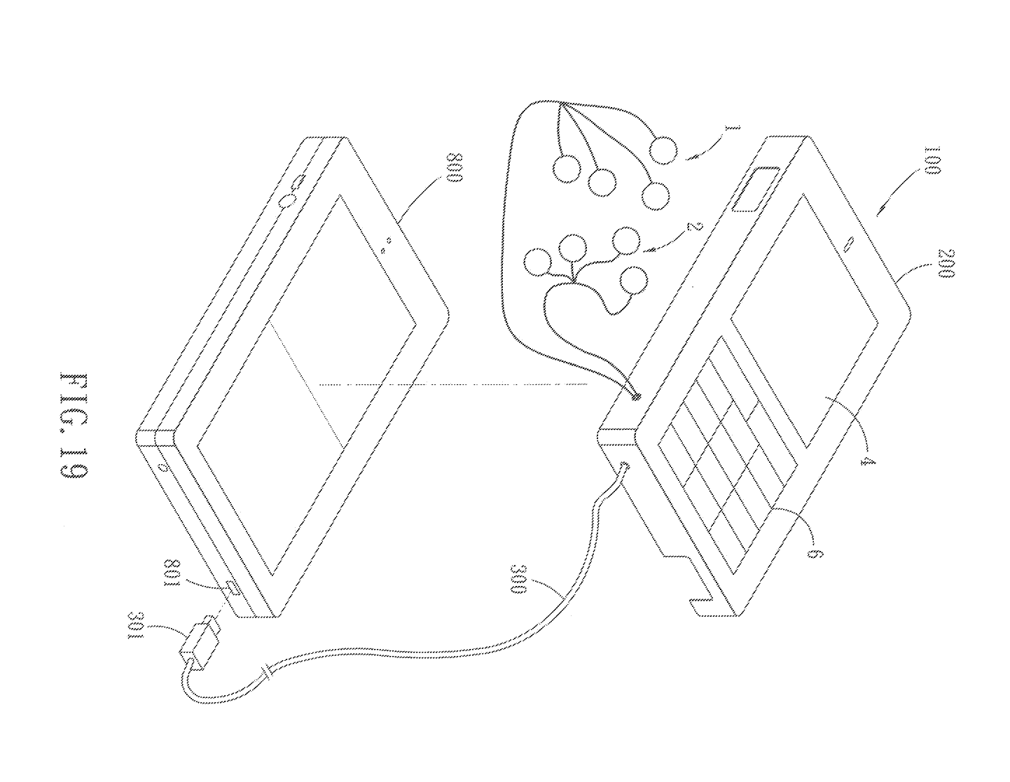

FIG. 3 to FIG. 6 illustrate respectively electromyograms of right-side orbicularis oris (OO) muscles, right-side masseter (MS) muscles, right-side submental (SUB) muscles and right-side laryngeal strap (LS) muscles of the subject with normal swallowing. FIG. 7 to FIG. 10 illustrate respectively electromyograms of left-side OO muscles, left-side MS muscles, left-side SUB muscles and left-side LS muscles of the subject with normal swallowing. FIG. 11 to FIG. 14 illustrate respectively electromyograms of right-side (i.e., healthy-side) OO muscles, right-side MS muscles, right-side SUB muscles and right-side LS muscles of the subject with swallowing disorder. FIG. 15 to FIG. 18 illustrate respectively electromyograms of left-side (i.e., unhealthy-side) OO muscles, left-side MS muscles, left-side SUB muscles and left-side LS muscles of the subject with swallowing disorder. Table 1 and Table 2 below illustrate the duration of muscle contraction (unit: sec) and the means of intensity (amplitude) of the electromyography signals (unit:μV) within the duration of muscle contraction associated with the swallowing muscles of a respective one of the subject with normal swallowing and the subject with swallowing disorder. Table 3 illustrates calculation results of the Pearson's correlation coefficients associated with the measured data.

TABLE 1

| Subject with normal swallowing | |
|---|---|
| Means | Duration of muscle |
| of intensity | contraction |

| | Right | Left | Right | Left |
|---|---|---|---|---|
| | side | side | side | side |
| OO muscles | 59.5143 | 63.8666 | 1.536 | 1.548 |
| MS muscles | 21.0977 | 24.8630 | 1.282 | 1.382 |
| SUB muscles | 20.0322 | 14.1143 | 1.242 | 1.318 |
| LS muscles | 12.7196 | 8.4068 | 1.083 | 1.26 |
| Average value | 28.5434 | 27.813 | 1.285 | 1.377 |

TABLE 2

| Subject with swallowing disorder | |
|---|---|
| Means | Duration of muscle |
| of intensity | contraction |

| | Right | Left | Right | Left |
|---|---|---|---|---|
| | side | side | side | side |
| OO muscles | 9.9774 | 6.86 | 1.846 | 0.624 |
| MS muscles | 27.3291 | 11.296 | 1.528 | 0.926 |
| SUB muscles | 10.2683 | 16.1691 | 0.82 | 0.802 |
| LS muscles | 8.8744 | 13.9671 | 0.96 | 0.888 |
| Average value | 14.1123 | 12.073 | 1.2885 | 0.810 |

TABLE 3

| | | Subject |
|---|---|---|
| Correlation between left | Subject | with |
| side data and right side | with normal | swallowing |
| data | swallowing | disorder |
| OO muscles integrated data | 0.9928 | 0.2022 |
| MS muscles integrated data | 0.9678 | 0.6962 |
| SUB muscles integrated data | 0.9741 | 0.9033 |
| LS muscles integrated data | 0.8936 | 0.9467 |
| Duration of muscle | 0.9862 | −0.5036 |
| contraction | | |
| Means of intensity | 0.9872 | −0.1355 |

It is evident from Table 3 that the calculation results of the correlations associated the subject with normal swallowing are mostly greater than 0.9, which implies that contraction situations between the left-side swallowing muscle group and the right-side swallowing muscle group resulting from swallowing behavior are similar. Table 3 further indicates that the subject with swallowing disorder has a relatively low correlation (0.2022) between the integrated data of a respective one of the unhealthy-side OO muscles and the healthy-side OO muscles, has a relatively low correlation (0.6962) between the integrated data of a respective one of the unhealthy-side MS muscles and the healthy-side MS muscles, has a relatively low correlation (−0.5036) between the duration of contraction of a respective one of the unhealthy-side swallowing muscle group and the healthy-side swallowing muscle group, and has a relatively low correlation (−0.1355) between the means of intensity of the electromyography signals of a respective one of the unhealthy-side swallowing muscle group and the healthy-side swallowing muscle group. It is apparent from the aforementioned data that through calculating the Pearson's correlation coefficients in this embodiment, severity of swallowing disorder of a subject may be evaluated.

A second preferred embodiment of the device for measuring and analyzing electromyography signals according to the present invention differs from the first preferred embodiment in the configuration that the number of the measuring electrodes of each of the unhealthy-side measuring electrode unit 1 and the healthy-side measuring electrode unit 2 is three. Each of the measuring electrodes of the corresponding measuring electrode unit may be disposed on one of: a respective one of the OO muscles, the MS muscles and the SUB muscles; a respective one of the OO muscles, the MS muscles and the LS muscles; and a respective one of the MS muscles, the SUB muscles and the LS muscles at the same side.

Referring to FIG. 19, the third preferred embodiment of the device 100 for measuring and analyzing electromyography signals according to the present invention is illustrated. In this embodiment, the device 100 further comprises a housing 200 for accommodating the control unit 3, the display unit 4, the wireless communication unit 5, the input unit 6 and the storage unit 7. The housing 200 is to be removably disposed on an external portable electronic device 800 (such as a mobile phone). More specifically, the housing 200 is designed to cover one side of the external portable electronic device 800 so as to achieve an effect of protecting the same. Furthermore, the device 100 for measuring and analyzing electromyography signals additionally comprises a transmission line 300 coupled electrically to the control unit 3. The transmission line 300 has a plug 301 to be connected to a transmission port 801 of the external portable electronic device 800, such that the control unit 3 is able to communicate with the external portable electronic device 800 via the transmission line 300. For instance, the measured data may be transmitted to the external portable electronic device 800. In another example, the control unit 3 is configured for communicating with the external portable electronic device 800 via the wireless communication unit 5 (see FIG. 1). By virtue of an integrated design of the device 100 and the housing 200, the device 100 for measuring and analyzing electromyography signals is easier to carry.

To sum up, the device 100 according to the present invention, by virtue of the non-invasive unhealthy-side measuring electrode unit 1 and the healthy-side measuring electrode unit 2 for measuring the electromyography signals of the target body part 9, the subject is not required to be exposed to radiation and experiences less discomfort during the assessment. It also takes shorter time to perform the assessment, and the device 100 has a relatively low cost and is easy to operate such that the device 100 of the present invention is more suitable for regular examination of a patient with swallowing disorder. Further, by virtue of the control unit 3 determining the correlation between the unhealthy-side measured data and the healthy-side measured data, severity of swallowing disorder of the subject may be evaluated based on the correlation.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A device for measuring and analyzing electromyography signals obtained from a target body part that is divided into an unhealthy-side muscle group and a healthy-side muscle group, said device comprising:
an unhealthy-side measuring electrode unit adapted to be disposed on the unhealthy-side muscle group and for measuring an electromyography signal from the unhealthy-side muscle group so as to generate unhealthy-side measured data;
a healthy-side measuring electrode unit adapted to be disposed on the healthy-side muscle group and for measuring an electromyography signal from the healthy-side muscle group so as to generate healthy-side measured data; and
a control unit connected electrically to said unhealthy-side measuring electrode unit and said healthy-side measuring electrode, and determining a correlation between the unhealthy-side measured data and the healthy-side measured data.

2. The device as claimed in claim 1, the unhealthy-side muscle group including unhealthy-side orbicularis oris (OO) muscles, the healthy-side muscle group including healthy-side orbicularis oris (OO) muscles, wherein:
said unhealthy-side measuring electrode unit includes a first unhealthy-side measuring electrode that is to be disposed on the unhealthy-side OO muscles and that is for measuring an electromyography signal thereof so as to generate first unhealthy-side measured sub-data of the unhealthy-side measured data;
said healthy-side measuring electrode unit includes a first healthy-side measuring electrode that is to be disposed on the healthy-side OO muscles and that is for measuring an electromyography signal thereof so as to generate first healthy-side measured sub-data of the healthy-side measured data; and
said control unit is configured to perform integration operations on the first unhealthy-side measured sub-data and the first healthy-side measured sub-data so as to obtain respectively first unhealthy-side integrated data and first healthy-side integrated data, and is further configured to determine a correlation between the first unhealthy-side integrated data and the first healthy-side integrated data.

3. The device as claimed in claim 1, the unhealthy-side muscle group including unhealthy-side masseter (MS) muscles, the healthy-side muscle group including healthy-side masseter (MS) muscles, wherein:
said unhealthy-side measuring electrode unit includes a second unhealthy-side measuring electrode that is to be disposed on the unhealthy-side MS muscles and that is for measuring an electromyography signal thereof so as to generate second unhealthy-side measured sub-data of the unhealthy-side measured data;
said healthy-side measuring electrode unit includes a second healthy-side measuring electrode that is to be disposed on the healthy-side MS muscles and that is for measuring an electromyography signal thereof so as to generate second healthy-side measured sub-data of the healthy-side measured data; and
said control unit is configured to perform integration operations on the second unhealthy-side measured sub-data and the second healthy-side measured sub-data so as to obtain respectively second unhealthy-side integrated data and second healthy-side integrated data, and is further configured to determine a correlation between the second unhealthy-side integrated data and the second healthy-side integrated data.

4. The device as claimed in claim 1, the unhealthy-side muscle group including unhealthy-side submental (SUB) muscles, the healthy-side muscle group including healthy-side submental (SUB) muscles, wherein:
said unhealthy-side measuring electrode unit includes a third unhealthy-side measuring electrode that is to be disposed on the unhealthy-side SUB muscles and that is for measuring an electromyography signal thereof so as to generate third unhealthy-side measured sub-data of the unhealthy-side measured data;
said healthy-side measuring electrode unit includes a third healthy-side measuring electrode that is to be disposed on the healthy-side SUB muscles and that is for measuring an electromyography signal thereof so as to generate third healthy-side measured sub-data of the healthy-side measured data; and
said control unit is configured to perform integration operations on the third unhealthy-side measured sub-data and the third healthy-side measured sub-data so as to obtain respectively third unhealthy-side integrated data and third healthy-side integrated data, and is further configured to determine a correlation between the third unhealthy-side integrated data and the third healthy-side integrated data.

5. The device as claimed in claim 1, the unhealthy-side muscle group including unhealthy-side laryngeal strap (LS) muscles, the healthy-side muscle group including healthy-side laryngeal strap (LS) muscles, wherein:
said unhealthy-side measuring electrode unit includes a fourth unhealthy-side measuring electrode than is to be disposed on the unhealthy-side LS muscles and that is for measuring an electromyography signal thereof so as to generate fourth unhealthy-side measured sub-data of the unhealthy-side measured data;
said healthy-side measuring electrode unit includes a fourth healthy-side measuring electrode that is to be disposed on the healthy-side LS muscles and that is for measuring an electromyography signal thereof so as to generate fourth healthy-side measured sub-data of the healthy-side measured data; and
said control unit is configured to perform integration operations on the fourth unhealthy-side measured sub-data and the fourth healthy-side measured sub-data so as to obtain respectively fourth unhealthy-side integrated data and fourth healthy-side integrated data, and is further configured to determine a correlation between the fourth unhealthy-side integrated data and the fourth healthy-side integrated data.

6. The device as claimed in claim 1, wherein:
said unhealthy-side measuring electrode unit includes a first unhealthy-side measuring electrode, a second unhealthy-side measuring electrode and a third unhealthy-side measuring electrode, each of which is for generating a respective one of first unhealthy-side measured sub-data, second unhealthy-side measured sub-data and third unhealthy-side measured sub-data of the unhealthy-side measured data;
said healthy-side measuring electrode unit includes a first healthy-side measuring electrode, a second healthy-side measuring electrode and a third healthy-side measuring electrode, each of which is for generating a respective one of first healthy-side measured sub-data, second healthy-side measured sub-data and third healthy-side measured sub-data of the healthy-side measured data; and
said control unit is configured to
calculate a mean of each of the first unhealthy-side measured sub-data, the second unhealthy-side measured sub-data, the third unhealthy-side measured sub-data, the first healthy-side measured sub-data, the second healthy-side measured sub-data and the third healthy-side measured sub-data, so as to generate a respective one of a first unhealthy-side measured mean, a second, unhealthy-side measured mean, a third unhealthy-side measured mean, a first healthy-side measured mean, a second healthy-side measured mean and a third healthy-side measured mean, and
determine a correlation between a set of the first unhealthy-side measured mean, the second unhealthy-side measured mean and the third unhealthy-side measured mean, and a set of the first healthy-side measured mean, the second healthy-side measured mean and the third healthy-side measured mean.

7. The device as claimed in claim 6, the unhealthy-side muscle group including unhealthy-side orbicularis oris (OO) muscles, unhealthy-side masseter (MS) muscles, unhealthy-side submental (SUB) muscles and unhealthy-side laryngeal strap (LS) muscles, the healthy-side muscle group including healthy-side orbicularis oris (OO) muscles, healthy-side masseter (MS) muscles, healthy-side submental (SUB) muscles and healthy-side laryngeal strap (LS) muscles, wherein:
each of said first unhealthy-side measuring electrode, said second unhealthy-side measuring electrode, said third unhealthy-side measuring electrode, said first healthy-side measuring electrode, said second healthy-side measuring electrode and said third healthy-side measuring electrode is to be disposed on one of
a respective one of the unhealthy-side OO muscles, the unhealthy-side MS muscles, the unhealthy-side SUB muscles, the healthy-side OO muscles, the healthy-side MS muscles and the healthy-side SUB muscles,
a respective one of the unhealthy-side OO muscles, the unhealthy-side MS muscles, the unhealthy-side LS muscles, the healthy-side OO muscles, the healthy-side MS muscles and the healthy-side LS muscles, and
a respective one of the unhealthy-side LS muscles, the unhealthy-side MS muscles, the unhealthy-side SUB muscles, the healthy-side LS muscles, the healthy-side MS muscles and the healthy-side SUB muscles.

8. The device as claimed in claim 6, wherein:
said unhealthy-side measuring electrode unit further includes a fourth unhealthy-side measuring electrode, which is for generating fourth unhealthy-side measured sub-data of the unhealthy-side measured data;
said healthy-side measuring electrode unit further includes a fourth healthy-side measuring electrode, which is for generating fourth healthy-side measured sub-data of the healthy-side measured data; and
said control unit is further configured to
calculate a mean of each of the fourth unhealthy-side measured sub-data and the fourth healthy-side measured sub-data, so as to generate a respective one of a fourth unhealthy-side measured mean and a fourth healthy-side measured mean, and
determine a correlation between a set of the first unhealthy-side measured mean, the second unhealthy-side measured mean, the third unhealthy-side measured mean and the fourth unhealthy-side measured mean, and a set of the first healthy-side measured mean, the second healthy-side measured mean, the third healthy-side measured mean and the fourth healthy-side measured mean.

9. The device as claimed in claim 8, the unhealthy-side muscle group including unhealthy-side orbicularis oris (OO) muscles, unhealthy-side masseter (MS) muscles, unhealthy-side submental (SUB) muscles and unhealthy-side laryngeal strap (LS) muscles, the healthy-side muscle group including healthy-side orbicularis oris (OO) muscles, healthy-side masseter (MS) muscles, healthy-side submental (SUB) muscles and healthy-side laryngeal strap (LS) muscles, wherein:
each of said first unhealthy-side measuring electrode, said second unhealthy-side measuring electrode, said third unhealthy-side measuring electrode, said fourth unhealthy-side measuring electrode, said first healthy-side measuring electrode, said second healthy-side measuring electrode, said third healthy-side measuring electrode and said fourth healthy-side measuring electrode is to be disposed on a respective one of the unhealthy-side OO muscles, she unhealthy-side MS muscles, the unhealthy-side SUB muscles, the unhealthy-side LS muscles, the healthy-side OO muscles, the healthy-side MS muscles, the healthy-side SUB muscles and the healthy-side LS muscles.

10. The device as claimed in claim 1, wherein:
said unhealthy-side measuring electrode unit includes a first unhealthy-side measuring electrode, a second unhealthy-side measuring electrode and a third unhealthy-side measuring electrode, each of which is for generating a respective one of first unhealthy-side measured sub-data, second unhealthy-side measured sub-data and third unhealthy-side measured sub-data of the unhealthy-side measured data;
said healthy-side measuring electrode unit includes a first healthy-side measuring electrode, a second healthy-side measuring electrode and a third healthy-side measuring electrode, each of which is for generating a respective one of first healthy-side measured sub-data, second healthy-side measured sub-data and third healthy-side measured sub-data of the healthy-side measured data; and
said control unit is configured to
generate a first unhealthy-side swallow duration value, a second unhealthy-side swallow duration value, a third unhealthy-side swallow duration value, a first healthy-side swallow duration value, a second healthy-side swallow duration value and a third healthy-side swallow duration value, each of which is associated with duration of muscle contraction due to swallowing behavior, according to the first unhealthy-side measured sub-data, the second unhealthy-side measured sub-data, the third unhealthy-side measured sub-data, the first healthy-side measured sub-data, the second healthy-side measured sub-data and the third healthy-side measured sub-data, respectively, and
determine a correlation between a set of the first unhealthy-side swallow duration value, the second unhealthy-side swallow duration value and the third unhealthy-side swallow duration value, and a set of the first healthy-side swallow duration value, the second healthy-side swallow duration value and the third healthy-side swallow duration value.

11. The device as claimed in claim 10, the unhealthy-side muscle group including unhealthy-side orbicularis oris (OO) muscles, unhealthy-side masseter (MS) muscles, unhealthy-side submental (SUB) muscles and unhealthy-side laryngeal strap (LS) muscles, the healthy-side muscle group including healthy-side orbicularis oris (OO) muscles, healthy-side masseter (MS) muscles, healthy-side submental (SUB) muscles and healthy-side laryngeal strap (LS) muscles, wherein:
each of said first unhealthy-side measuring electrode, said second unhealthy-side measuring electrode, said third unhealthy-side measuring electrode, said first healthy-side measuring electrode, said second healthy-side measuring electrode and said third healthy-side measuring electrode is to be disposed on one of
a respective one of the unhealthy-side OO muscles, the unhealthy-side MS muscles, the unhealthy-side SUB muscles, the healthy-side OO muscles, the healthy-side MS muscles and the healthy-side SUB muscles,
a respective one of the unhealthy-side OO muscles, the unhealthy-side MS muscles, the unhealthy-side LS muscles, the healthy-side OO muscles, the healthy-side MS muscles and the healthy-side LS muscles, and
a respective one of the unhealthy-side LS muscles, the unhealthy-side MS muscles, the unhealthy-side SUB muscles, the healthy-side LS muscles, the healthy-side MS muscles and the healthy-side SUB muscles.

12. The device as claimed in claim 10, wherein:
said unhealthy-side measuring electrode unit further includes a fourth unhealthy-side measuring electrode, which is for generating fourth unhealthy-side measured sub-data of the unhealthy-side measured data;
said healthy-side measuring electrode unit further includes a fourth healthy-side measuring electrode, which is for generating fourth healthy-side measured sub-data of the healthy-side measured data; and
said control unit is further configured to
generate a fourth unhealthy-side swallow duration value and a fourth healthy-side swallow duration value, each of which is associated with duration of muscle contraction due to swallowing behavior, according to the fourth unhealthy-side measured sub-data and the fourth healthy-side measured sub-data, respectively, and
determine a correlation between a set of the first unhealthy-side swallow duration value, the second unhealthy-side swallow duration value, the third unhealthy-side swallow duration value and the fourth unhealthy-side swallow duration value, and a set of the first healthy-side swallow duration value, the second healthy-side swallow duration value, the third healthy-side swallow duration value, and the fourth healthy-side swallow duration value.

13. The device as claimed in claim 12, the unhealthy-side muscle group including unhealthy-side orbicularis oris (OO) muscles, unhealthy-side masseter (MS) muscles, unhealthy-side submental (SUB) muscles and unhealthy-side laryngeal strap (LB) muscles, the healthy-side muscle group including healthy-side orbicularis oris (OO) muscles, healthy-side masseter (MS) muscles, healthy-side submental (SUB) muscles and healthy-side laryngeal strap (LS) muscles, wherein:
each of said first unhealthy-side measuring electrode, said second unhealthy-side measuring electrode, said third unhealthy-side measuring electrode, said fourth unhealthy-side measuring electrode, said first healthy-side measuring electrode, said second healthy-side measuring electrode, said third healthy-side measuring electrode and said fourth healthy-side measuring electrode is to be disposed on a respective one of the unhealthy-side OO muscles, the unhealthy-side MS muscles, the unhealthy-side SUB muscles, the unhealthy-side LS muscles, the healthy-side OO muscles, the healthy-side MS muscles, the healthy-side SUB muscles and the healthy-side LS muscles.

14. The device as claimed in claim 1, further comprising a housing for accommodating said control unit.

15. The device as claimed in claim 14, wherein said housing is to be removably disposed on an external portable electronic device, and said control unit is configured for communicating with the external portable electronic device in a wired manner.

16. The device as claimed in claim 14, wherein said housing is to be removably disposed on an external portable electronic device, and said control unit is configured for communicating with the external portable electronic device in a wireless manner.

17. The device as claimed in claim 1, further comprising a wireless communication unit coupled to said control unit for communicating wirelessly with an external portable electronic device.
